(19) United States
(12) Patent Application Publication
(10) Pub. No.: US 2021/0100630 A1
(43) Pub. Date: Apr. 8, 2021

(54) MEDICAL WASTE MANAGEMENT DRAPE

(71) Applicant: Sandia Surgical Innovations LLC, Albuquerque, NM (US)

(72) Inventors: Kelly Rivera, Albuquerque, NM (US); Michel Tucker, Albuquerque, NM (US)

(21) Appl. No.: 17/063,481

(22) Filed: Oct. 5, 2020

Related U.S. Application Data
(60) Provisional application No. 62/911,235, filed on Oct. 5, 2019.
(60) Provisional application No. 62/987,768, filed on Mar. 10, 2020.

(51) Int. Cl. A61B 46/23

(52) U.S. Cl. CPC A61B 46/23; A61B 2217/005; A61B 2046/236

(57) ABSTRACT

The systems and methods disclosed herein comprise an absorbent pad, at least one perforation formed in the absorbent pad configured to fit a medical device, and at least one drape flap attached to an end of the absorbent pad, the absorbent pad further comprising a top layer and a bottom layer, and an absorbent layer contained between the top layer and bottom layer.

### CROSS REFERENCE TO RELATED APPLICATIONS

This patent application claims the priority and benefit under 35 U.S.C. § 119(e) of U.S. Provisional Patent Application Ser. No. 62/911,235, filed Oct. 5, 2019, entitled “MEDICAL WASTE MANAGEMENT DRAPE.” U.S. Provisional Patent Application Ser. No. 62/911,235 is herein incorporated by reference in its entirety. This patent application also claims the priority and benefit under 35 U.S.C. § 119(e) of U.S. Provisional Patent Application Ser. No. 62/987,768, filed Mar. 10, 2020, entitled “MEDICAL WASTE MANAGEMENT DRAPE.” U.S. Provisional Patent Application Ser. No. 62/987,768 is herein incorporated by reference in its entirety.

### TECHNICAL FIELD

Embodiments disclosed herein are generally related to medical devices. Embodiments are additionally related to medical drapes. Embodiments are also related to medical drapes configured to collect fluid. Embodiments are further related to sterile medical coverings for surgical applications, where the coverings are configured to collect medical waste.

### BACKGROUND

It is standard medical practice to maintain a sterile operating environment. Surgeons, nurses, and other hospital staff go to great lengths to ensure the surgical instruments, and the field in which they operate, are kept sterile. Medical professionals are diligent in managing the sterility of the operating environment.

Certain medical operations, particularly surgical operations, may result in significant medical waste. For example, orthopedic operations often require incisions that result in the discharge of large amounts of blood and other bodily fluids, draining from the incision during the procedure. Additionally, many operations require copious irrigation with sterile fluid, which produces large amounts of fluidic waste, some or all of which may be a biohazard.

This medical fluid can create significant problems. First, in cases where large amounts of fluid are released, the drainage has to be collected, managed, and/or cleaned as the operation proceeds. This requires critical attention from medical staff who could otherwise be attending to other matters. There is no currently available solution to this problem.

In addition, most medical waste fluid is generally sterile as it exits the body, but may also be a biohazard, and/or become unsterile after it comes in contact with other objects in the room. Thus, it is a medical necessity to avoid contact between medical waste and other objects in the operating room to avoid any possibility of cross-contamination.

Furthermore, large volumes of fluid, including biological material and wound irrigation fluid, ends up on the floor during an operation. The resulting slippery surfaces create a high risk of work related injuries if the liquid on the floor is not managed.

Current solutions generally involve the use of sterile wipes or rags. Multiple rags can be used to collect the fluid as it drains. However, these rags are generally not configured or shaped to interface with the various other medical devices associated with the medical procedure. Positioning, monitoring, and accounting for various towels and other surgical equipment requires significant effort and attention from staff who might otherwise be able to provide care to the patient.

As such, there is a need in the art for medical waste collection drapes as disclosed in the embodiments detailed herein.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, an aspect of the disclosed embodiments to provide medical devices.

In addition, it is another aspect of the embodiments to provide surgical equipment.

It is another aspect of the disclosed embodiments to provide methods and systems for collecting medical fluid during surgical operations.

The aforementioned aspects and other objectives and advantages can now be achieved as described herein. In one embodiment, a system comprises an absorbent pad, at least one perforation formed in the absorbent pad configured to fit a medical device, and at least one drape flap attached to an end of the absorbent pad. In an embodiment, the absorbent pad further comprises a top layer and a bottom layer, and an absorbent layer contained between the top layer and bottom layer. In an embodiment, the top layer further comprises a permeable material. In an embodiment, the absorbent layer further comprises at least one of: a network of fluidic channels and an absorbent material. In an embodiment, the system further comprises a drain connected to the network of fluidic channels. In an embodiment, the system further comprises a connection port associated with the drain and configured to engage with a suctioning device.

In another embodiment, a surgical drape waste management system comprises a drape body, at least one perforation formed in the drape body, a first drape flap, a second drape flap, and a raised border surrounding at least one side of the drape body. In an embodiment, the drape body further comprises a top layer, a bottom layer, and an intermediate layer configured between the top layer and the bottom layer.

In an embodiment the top layer further comprises a permeable material. In an embodiment, the intermediate layer further comprises a ductwork of fluidic channels. In an embodiment, the ductwork of fluidic channels further comprises a ductwork of microfluidic channels integrated in a fabric. In an embodiment, the ductwork of fluidic channels further comprises perforations on an upward facing portion of the microfluidic channels. In an embodiment, the surgical drape waste management system further comprises a drain connected to the ductwork of microfluidic channels. In certain embodiments, the intermediate layer further comprises an absorbent material.

In another embodiment of the surgical drape waste management system, the bottom layer further comprises a water resistant sheet of material with an outward facing high friction coating on the bottom layer.

In an embodiment, the surgical drape waste management system further comprises at least one connector formed on the first drape flap and at least one connector formed on the second drape flap.

In yet another embodiment, a medical drape comprises a drape body, at least one perforation formed in the drape body, a first drape flap, at least one connector formed on the first drape flap, a second drape flap, and at least one connector formed on the second drape flap. In an embodiment, each of the at least one connectors comprise hook and loop fasteners. In an embodiment, the at least one perforation comprises one of a removable rectangular outline formed in the drape body, and at least two stitched slits formed in the drape body.

### BRIEF DESCRIPTION OF THE FIGURES

The accompanying figures, in which like reference numerals refer to identical or functionally-similar elements throughout the separate views and which are incorporated in, and form a part of the specification, further illustrate the embodiments and, together with the detailed description, serve to explain the embodiments disclosed herein.

FIG. 1 is a prior art depiction of a surgical positioning system associated with the disclosed embodiments;

FIG. 2 depicts a medical waste management drape, in accordance with the disclosed embodiments;

FIG. 3 depicts a flow chart of steps associated with a method of using a medical waste management drape, in accordance with the disclosed embodiments;

FIG. 4 depicts another embodiment of a medical waste management drape, in accordance with the disclosed embodiments;

FIG. 5 depicts another embodiment of a medical waste management drape, in accordance with the disclosed embodiments;

FIG. 6 depicts another embodiment of a medical waste management drape, in accordance with the disclosed embodiments; and

FIG. 7 depicts an embodiment of a low cost medical waste management drape, in accordance with the disclosed embodiments.

### DETAILED DESCRIPTION

The particular values and configurations discussed in the following non-limiting examples can be varied, and are cited merely to illustrate one or more embodiments and are not intended to limit the scope thereof.

Example embodiments will now be described more fully hereinafter with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood the principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, Aft AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, Aft BBC, AAABCCCC, CBBAAA, CABABB, and so forth. The skilled artisan will understand that typically there is no limit on the number of items or terms in any combination, unless otherwise apparent from the context. All aspects of the various embodiments, can be interchangeable with aspects disclosed in other embodiments, and/or can be incorporated in other embodiments.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

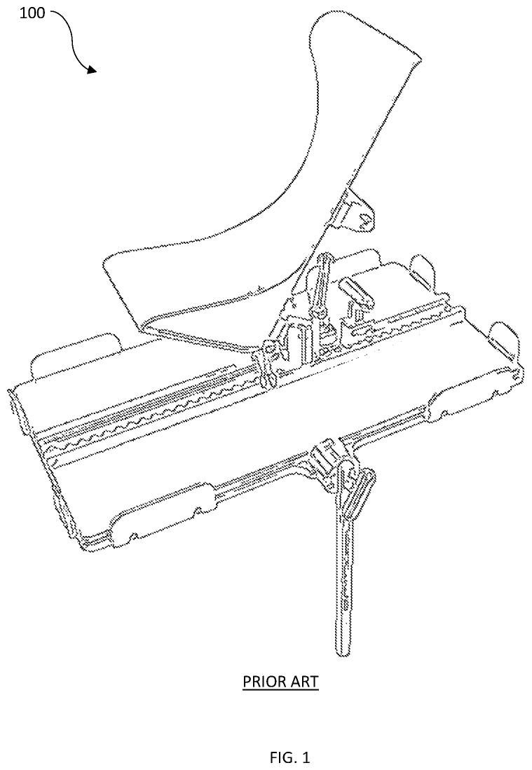

Surgical procedures often require specialized equipment used to position the patient's body, or body parts, for the operation. FIG. 1 illustrates one such device 100, labeled prior art, that is commonly used for certain knee surgeries. The base of the device sits on the operating table and includes a stepped mechanism. With the patient lying on their back, their foot can be positioned in the foot holster. The stepped mechanism can then be used to adjust the bend in the patient's knee as required for the procedure.

The device 100 is critical for positioning the patient knee. However, most knee operations, and other such operations, involve a significant amount of fluid drainage and waste. With the device 100 positioned on the operating table it is difficult to manage such drainage with standard operating towels and drapes, while maintaining a sterile environment. FIG. 1, illustrates one such device 100, but there are many other, similar surgical positioning devices, all of which create a similar problem.

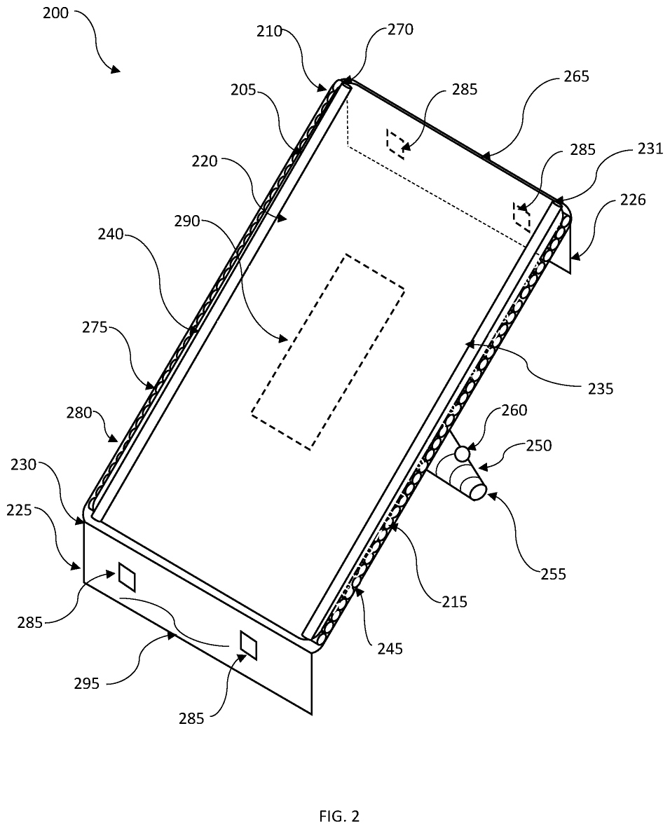

The embodiments disclosed herein include a method and system for collecting medical waste with a specially designed surgical drape. FIG. 2 illustrates an exemplary embodiment of a surgical drape waste management system 200. Surgical drape waste management system 200 is generally configured with a top layer 205 and a bottom layer 210. An intermediate layer 215 can be formed between the top layer 205 and bottom layer 210. In certain embodiments, the edges of top layer 205 and bottom layer 210 can be bound to enclose intermediate layer 215 there between. This can be accomplished with stitching, adhesive, heat treatment or other such means of connecting material.

The two ends of the surgical drape waste management system 200 include seam 230 and seam 231 that connect the surgical drape waste management system's main drape body 220 to a first drape flap 225 and a second drape flap 226 respectively. In other embodiments, the surgical drape waste management system 200 can have seams attached to drape flaps on all the sides. In certain embodiments, additional flaps or connections can be formed of waste management materials so that it can be laid flat or used to wrap around a surgical device such as a knee positioning device 100. As such, in certain embodiments the drape flaps can include a top layer, intermediate layer and bottom layer as in the main body 220, or can be configured as a single layer of material.

Each of the layers and components of the surgical drape waste management system 200 can be sterilized and/or formed of sterile materials, sufficient that the system can be used in operating rooms or other applications where a sterile environment is required.

The surgical drape waste management system 200 can generally be rectangular in shape. However, in other embodiments other shapes may also be used as required for the specific operational application. For example, in certain embodiments, the surgical drape waste management system 200 can be circular, ovular, triangular, square, etc.

The top layer 205, of the surgical drape waste management system 200 can be a specially configured material designed to collect medical fluid. In certain embodiments, this material can be selected accordingly. The top layer 205 can generally be configured to catch medical waste and prevent it from draining onto surrounding medical equipment, or other areas of the operating room, but also allow the medical waste to pass through the top layer to the intermediate layer covered by the top layer. The material can further be selected to be a fabric so that it can be arranged to fit in various operating rooms and/or around various medical devices such as device 100.

In certain embodiments, the top layer 205 can be surrounded by a raised border 235, which can comprise a border, lip, and/or perimeter of liquid absorbing material. The raised border 235 can prevent excess fluid collected on the top layer 205 from spilling onto the floor or surrounding equipment. In certain embodiments, the raised border 235 can be stitched into a casing 240 surrounding, and generally on, the top layer 205. In other embodiments, the absorbent material can be directly connected to the top layer 205.

The intermediate layer 215 can comprise one or more materials. In one embodiment, the intermediate layer can comprise a ductwork of fluidic channels 245. The fluidic channels 245 can be configured of plastic, polymer, or other such lightweight material. The fluidic channels 245 can comprise microfluidic channels woven into the material of the intermediate layer 215. The fluidic channels 245 can be perforated tubing, with perforations formed on the upward facing sides of the fluidic channels, so that medical liquid draining through the top layer collects in the fluidic channels.

The channels can comprise multiple interconnected or independent ducts, all of which can connect to a drain 250. The drain 250 can include a connection port 255 that can connect to a suction device so that liquid collected in the fluidic channels can be drawn out of the intermediate layer 215 into a collection receptacle via negative pressure (i.e. suction) provided by a suction device connected to the suction connection port 255. The connection port 255 can be configured with a valve 260 that allows the port to be opened and closed as necessary.

In certain embodiments, a perimeter or raised border 265 can be formed along the edge of the top layer 205. The raised border 265 can comprise perforations forming fluidic access to suction tubing 270 internal to the raised border 265. The perforated suction tubing 270 can include perforations on the inward facing edges of the raised border 265. The suction tubing can be fluidically connected to the fluidic channels 245, and by extension, the suction device via port 255. As the fluid pools on the top surface 205, the suction tubing 207 can be used to suck the fluid into the tubing where it can be collected in a collection receptacle.

The intermediate layer 215 can further include absorbent material 275 or a combination of materials such as cotton, rayon, sodium polyacrylate, liquid retaining gel, potassium polyacrylate and other synthetic materials configured to absorb medical fluid that passes through the top layer 205. The absorbent material 275 can comprise packing material that surrounds the fluidic channels 245 formed in the intermediate layer 215 and/or in the top layer 205. It should be appreciated that, in embodiments where channels are not included in the intermediate layer 215, the intermediate layer 215 can comprise some or all of the absorbent material(s) 275.

The bottom layer 210 can comprise a medical grade water resistant cover 280 that encloses the intermediate layer 215 between the top layer 205 and bottom layer 210. The bottom layer 210 (or cover) is selected to be water resistant to prevent any fluid that passes through the top layer 205 and intermediate layer 215 from draining through the drape and spilling in the operating room or surgical environment.

The bottom cover 280 can comprise an outward facing (or downward facing) layer that is coated in a high friction coating such as stickem, adhesive, or the like, to prevent the system 200 from sliding out of place during use. In other embodiments, gripping surfaces with a high coefficient of friction can be configured at various locations on the outward facing (or downward facing) side of the bottom cover 280. The top layer 205 can be connected to the bottom layer 210 with stitching or seams to enclose the intermediate layer 215 therebetween.

Each end of the surgical drape waste management system 200 can be connected to drape flap 225 and drape flap 226. The drape flaps 225 and 226 can be stitched or seamed to the main body 220 of the surgical drape waste management system 200. Each of drape flap 225 and drape flap 226 can be configured to be a single ply sheet of sterile material, or can include one or more of the top layer 205, intermediate layer 215, and/or bottom layer 210 materials. The drape flap seams 230 and 231 can have a perimeter of tubular sponge like material to collect and/or retain excess fluid on the top of the surgical drape waste management system 200.

An arrangement of one or more connecting members 285 can be configured in, or on each of the drape flaps 225 and 226. The connecting members 285 can comprise an outward and/or inward facing sticking material, adhesive, glue, magnets or the like. In other embodiments, the connectors 285 can comprise other such connecting means.

The drape flaps 225 and 226 are configured to wrap below a surgical device, such as device 100, operating table, or other such surgical system. The drape flaps 225 and 226 can include interfacing connecting points that allow one drape flap, for example drape flap 225 to engage to another drape flap, such as drape flap 226. The connecting members 285 can connect the drape flap 225 and drape flap 226 to the underside of the surgical device, such as device 100, to hold the surgical drape waste management system 200 in place.

It should be appreciated that in FIG. 2, two connecting members 285 are illustrated on each of the drape flap 225 and drape flap 226. However, in other embodiments, more or fewer connecting members 285 can be formed in the drape flaps without departing from the scope of the embodiments disclosed herein.

The surgical drape waste management system 200 can further include perforations 290 (which may also be embodied as cutouts, seams, slits, or the like) formed in the center of the body 220 of the surgical drape waste management system 200. The perforations 290 are configured to allow the surgical drape waste management system to be installed in or on medical devices used during surgical procedures.

For example, in an embodiment, perforations formed in the surgical drape waste management system can be configured so that the surgical drape waste management system can be placed on top of the sterile tray and below the leg holding mechanism of a surgical knee holder device such as device 100, or other such device. The perforations 290 allow a section of the body 220 of the surgical drape waste management system 200 to be removed. The user can elect to remove the entire section or can separate some, but not all, of the perforated material or section to conform with or around the necessary medical device.

In another embodiment, a barium sulfate band 295 can be woven into one or both of drape flap 225 and drape flap 226, which can be provided to improve X-ray detection.

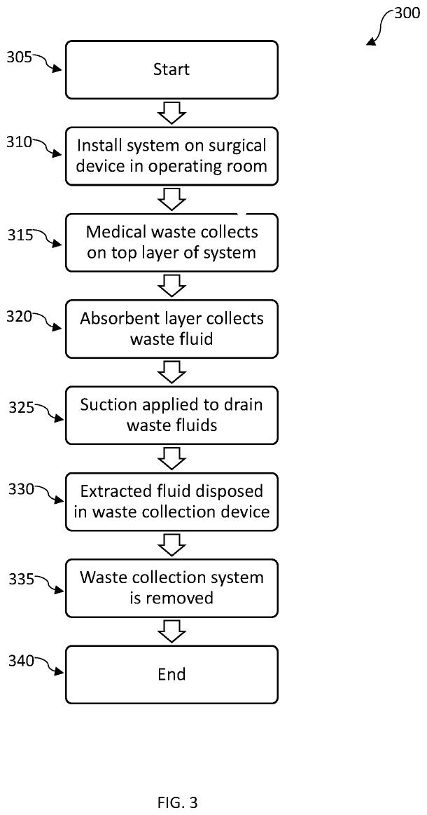

FIG. 3 illustrates an exemplary method 300 in accordance with the disclosed embodiments. The method starts at 305.

At step 310 a surgical drape waste management system 200 can be in installed on or around a surgical device, such as device 100, table, operating table, or the like. For instance, in an exemplary embodiment, the surgical drape waste management system 200 can be connected on, or around, an surgical knee holder device. The perforations 290 can be used to remove the necessary portion of the surgical drape waste management system 200, to fit around or over the device. As medical waste fluid drains during the medical procedure, it can collect on the top layer 205 of the surgical drape waste management system 200 as shown at step 315.

Medical fluid that collects on the top layer 205 can permeate through the top layer 205 into the absorbent layer or intermediate layer 215 of the surgical drape waste management system 200. The absorbent layer 215 collects the medical fluid and waste at step 320. As the absorbent material collects the medical fluid, a suction device can be used to extract the fluid that is collected in the intermediate layer 215, in absorbent material and/or in the fluidic channels 245 formed in the intermediate layer 215 as shown at step 325. As the fluid is extracted, it can be disposed in a waste collection receptacle at step 330.

Once the surgical procedure is complete, the surgical drape waste management system 200 can be removed at step 335 at which point the method is completed at step 340.

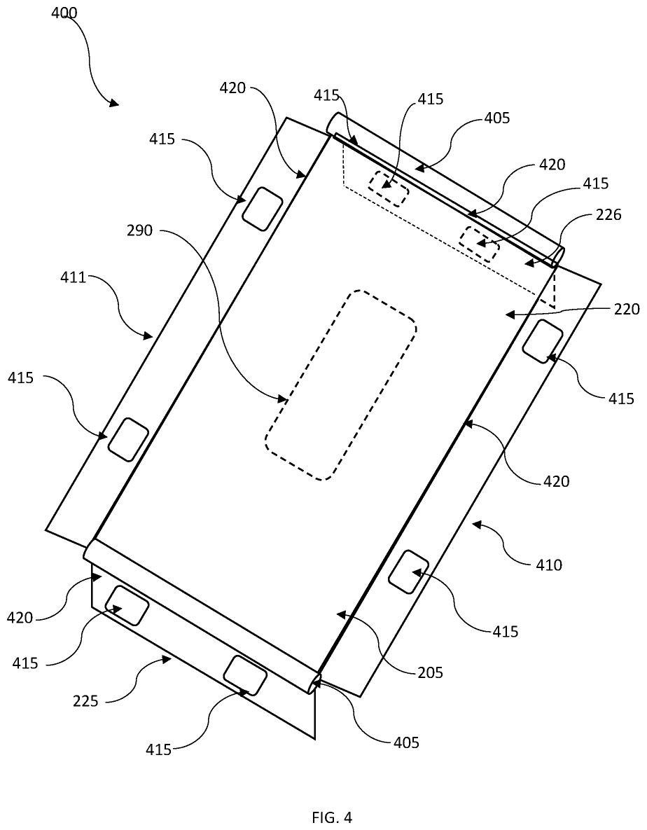

FIG. 4 illustrates another embodiment of a surgical drape waste management system 400. It should be appreciated that some or all of the features illustrated in FIG. 2 can be incorporated in the embodiments illustrated in FIG. 4. Some or all of those features may share reference numbers.

The surgical drape waste management system 400, illustrated in FIG. 4 can include an absorbent lip or rim 405 that can comprise a sponge, or other highly absorbent material formed along the perimeter and/or edge of the top layer 205. The absorbent lip or rim 405 can be connected to the top layer 205 with stitching, seams, or perforations 420. In certain embodiments, the absorbent lip 405 can be connected with a hook and loop connector 415 or other such fastening means so that it can optionally be removed and discarded. In FIG. 4 the lip 405 is shown on two sides, but it should be appreciated that in other embodiments it can be further configured on all sides of the system 400 to form a perimeter around the top layer 205.

FIG.4 further illustrates a system with four drape flaps, drape flaps 225 and 226, along with side drape flap 410 and side drape flap 411 connected to each edge of the main body 220. The drape flaps 225 and 226, and side drape flaps 410 and 411 can each have one or more connectors 415. In certain, embodiments, the connectors 415 can comprise magnets or stickies with peel-off covers formed on the drape flap connectors 415. The perforations 290 in the center of the body 220 can be a full rectangle, circular, ovular, can include nested perforations, can be a single line or slit, or two parallel lines. The drape material or intermediate layer 215, can comprise sodium polyacrylate or SOCO polymer.

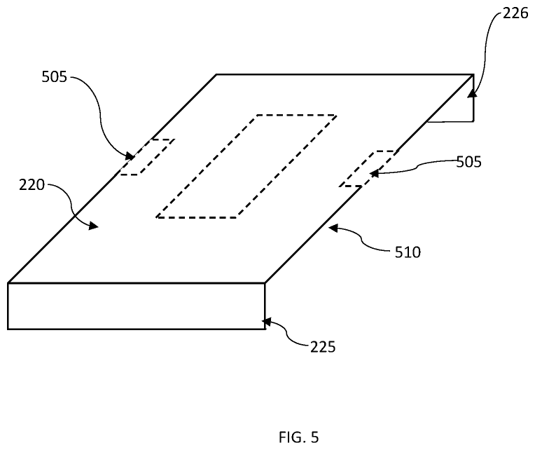

FIG. 5 illustrates another embodiment of a surgical drape waste management system 500. It should be appreciated that some or all of the features illustrated in FIGS. 2 and 4 can be incorporated in the embodiments illustrated in FIG. 5. Some or all of those features may share reference numbers. In the embodiment illustrated in FIG. 5, perforations 505 are provided in or along the edges of the main body of the system. The perforations 505 allow a section of the main body 220 to be removed so that a clamp can be connected, such as a clamp used with certain knee positioner devices. The outward surface 510 of the bottom layer of the drape system 500 can be coated with a sticky substance to prevent the system from moving. In addition, the system can be configured with a void (or perforations for removal of a section) in the center of the main body that allows the system to be placed on or around a medical device, or surgical apparatus.

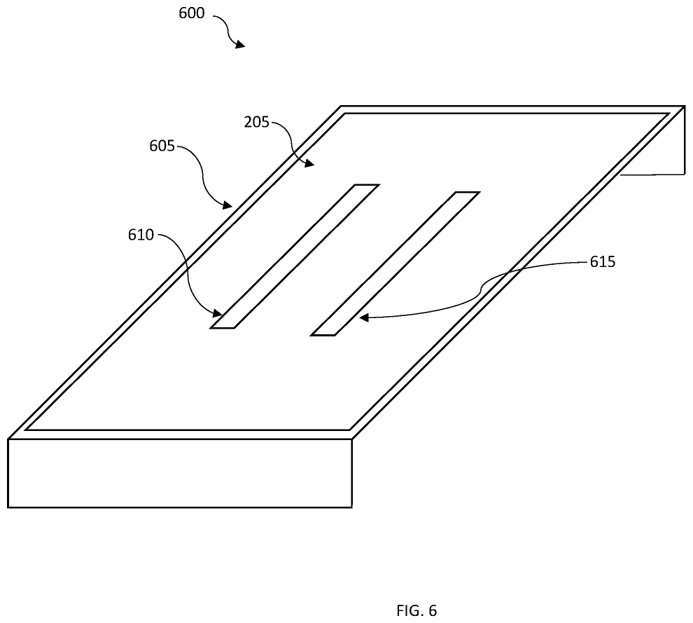

FIG. 6 illustrates another embodiment of a surgical drape waste management system 600. It should be appreciated that some or all of the features illustrated in FIGS. 2, 4, and 5 can be incorporated in the embodiment illustrated in FIG. 6, in which a plastic lip 605 can be configured around the top layer 205 to prevent liquid that collects thereon from spilling. The lip 605 can be formed of plastic, rubber, metal, or other such sterile material. In certain embodiments, no perforations can be formed in the system 600.

FIG. 6 further illustrates an arrangement that includes a first slit 610 and a second parallel slit 615. The first slit 610 can comprise a stitched slit and the second slit 615 can also include a stitched slit. The stitched slits that allow the system to be fit over specific medical or surgical equipment.

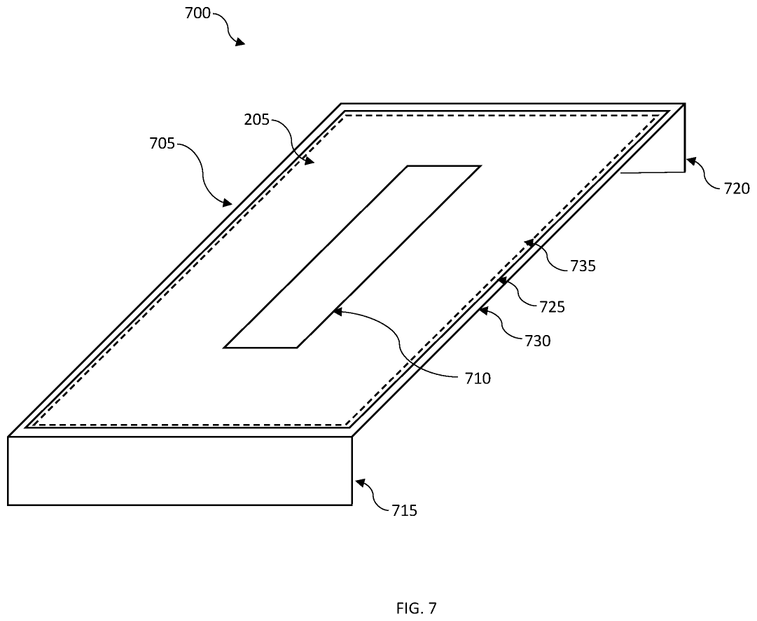

FIG. 7 illustrates a low cost surgical drape 700. In an embodiment, the surgical drape can comprise an absorbent pad 705, and one or more perforations 710 formed in the absorbent pad 705. The perforation can be configured to allow a section of the absorbent pad to be removed so that the absorbent pad can fit around or over a medical positioning device such as the device 100 illustrated in FIG. 1. The surgical drape 700 can further comprise drape flap 715 and drape flap 720 attached to an end of the absorbent pad 705. In the embodiment, illustrated in FIG. 7, the absorbent pad 705 can comprise a permeable top layer 725, and an impermeable bottom layer 730 which enclose an intermediate absorbent layer 735. The low cost embodiment illustrated in FIG. 7, can comprise a sterile, disposable system for use in managing medical waste during medical procedures.

It should be noted that the disclosed systems and methods are configured to ensure medical fluid and waste, common during many surgical procedures, does not leak or spill into the surrounding environment. The embodiments include specific features such that the systems and methods neatly interface with the operating tools. In specific embodiments, the systems and methods can be used in conjunction with devices such as the knee bending device 100 shown in FIG. 1. The embodiments disclosed herein, including the surgical drape waste management system, are generally intended to be disposable after use, but in certain embodiments, they can also be configured to be washed, sterilized and reused.

Based on the foregoing, it can be appreciated that a number of embodiments, preferred and alternative, are disclosed herein. For example, in one embodiment a system comprises an absorbent pad, at least one perforation formed in the absorbent pad, the at least one perforation configured to fit a medical device, and at least one drape flap attached to an end of the absorbent pad. In an embodiment, the absorbent pad further comprises a top layer and a bottom layer, and an absorbent layer contained between the top layer and bottom layer. In an embodiment, the top layer further comprises a permeable material. In an embodiment, the absorbent layer further comprises at least one of: a network of fluidic channels and an absorbent material. In an embodiment, the system further comprises a drain connected to the network of fluidic channels. In an embodiment, the system further comprises a connection port associated with the drain and configured to engage with a suctioning device.

In another embodiment, a surgical drape waste management system comprises a drape body, at least one perforation formed in the drape body, a first drape flap, a second drape flap, and a raised border surrounding at least one side of the drape body. In an embodiment, the drape body further comprises a top layer, a bottom layer, and an intermediate layer configured between the top layer and the bottom layer.

In an embodiment the top layer further comprises a permeable material. In an embodiment, the intermediate layer further comprises a ductwork of fluidic channels. In an embodiment, the ductwork of fluidic channels further comprises a ductwork of microfluidic channels integrated in a fabric. In an embodiment, the ductwork of fluidic channels further comprises perforations on an upward facing portion of the microfluidic channels. In an embodiment, the surgical drape waste management system further comprises a drain connected to the ductwork of microfluidic channels. In certain embodiment, the intermediate layer further comprises: an absorbent material.

In another embodiment of the surgical drape waste management system the bottom layer further comprises a water resistant sheet of material with an outward facing high friction coating on the bottom layer.

In an embodiment, the surgical drape waste management system further comprises at least one connector formed on the first drape flap and at least one connector formed on the second drape flap.

In yet another embodiment, a medical drape comprises a drape body, at least one perforation formed in the drape body, a first drape flap, at least one connector formed on the first drape flap, a second drape flap, and at least one connector formed on the second drape flap. In an embodiment, each of the at least one connectors comprise hook and loop fasteners. In an embodiment, the at least one perforation comprises one of a removable rectangular outline formed in the drape body, and at least two stitched slits formed in the drape body.

It should be appreciated that variations of the above-disclosed and other features and functions, or alternatives thereof, may be desirably combined into many other different systems or applications. It should be understood that various presently unforeseen or unanticipated alternatives, modifications, variations or improvements therein may be subsequently made by those skilled in the art which are also intended to be encompassed by the following claims.

What is claimed is:

1. A system comprising:
an absorbent pad;
at least one perforation formed in the absorbent pad the at least one perforation configured to fit a medical device; and
at least one drape flap attached to an end of the absorbent pad.

2. The system of claim 1 wherein the absorbent pad further comprises:
a top layer and a bottom layer; and
an absorbent layer contained between the top layer and the bottom layer.

3. The system of claim 2 wherein the top layer further comprises:
a permeable material.

4. The system of claim 2 wherein the absorbent layer further comprises at least one of:
a network of fluidic channels; and
an absorbent material.

5. The system of claim 4 further comprising:
a drain connected to the network of fluidic channels.

6. The system of claim 5 further comprising:
a connection port associated with the drain and configured to engage with a suctioning device.

7. A surgical drape waste management system comprising:
a drape body;
at least one perforation formed in the drape body;
a first drape flap;
a second drape flap; and
a raised border surrounding at least one side of the drape body.

8. The surgical drape waste management system of claim 7 wherein the drape body further comprises:
a top layer;
a bottom layer; and
an intermediate layer configured between the top layer and the bottom layer.

9. The surgical drape waste management system of claim 8 where in the top layer further comprises:
a permeable material.

10. The surgical drape waste management system of claim 8 wherein the intermediate layer further comprises:
a ductwork of fluidic channels.

11. The surgical drape waste management system of claim 10 wherein the ductwork of fluidic channels further comprises:
a ductwork of microfluidic channels integrated in a fabric.

12. The surgical drape waste management system of claim 11 wherein the ductwork of fluidic channels further comprises:
perforations on an upward facing portion of the microfluidic channels.

13. The surgical drape waste management system claim 11 further comprising:
a drain connected to the ductwork of microfluidic channels.

14. The surgical drape waste management system of claim 8 wherein the intermediate layer further comprises:
an absorbent material.

15. The surgical drape waste management system of claim 8 wherein the bottom layer further comprises:
a water resistant sheet of material.

16. The surgical drape waste management system of claim 15 further comprising:
an outward facing high friction coating on the bottom layer.

17. The surgical drape waste management system of claim 8 further comprising:
at least one connector formed on the first drape flap; and
at least one connector formed on the second drape flap.

18. A medical drape comprising:
drape body;
at least one perforation formed in the drape body;
a first drape flap;
at least one connector formed on the first drape flap;
a second drape flap; and
at least one connector formed on the second drape flap.

19. The medical drape of claim 18 wherein each of the at least one connectors comprise hook and loop fasteners.

20. The medical drape of claim 18 wherein the at least one perforation comprises one of:
a removable rectangular outline formed in the drape body; and
at least two stitched slits formed in the drape body.
